(19) United States
(12) Patent Application Publication
(10) Pub. No.: US 2013/0123298 A1
(43) Pub. Date: May 16, 2013

(54) PHARMACEUTICAL COMPOSITION FOR THE PREVENTION OF PERIOPERATIVE ARTERIAL HYPOTENSION IN HUMANS

(71) Applicant: Jean-Michel Julia, Montpellier (FR)

(72) Inventor: Jean-Michel Julia, Montpellier (FR)

(73) Assignee: LABORATOIRE AGUETTANT, Lyon, FR (FR)

(21) Appl. No.: 13/812,206

(22) PCT Filed: Jul. 27, 2011
(86) PCT No.: PCT/FR11/51803

§ 371 Date: Jan. 25, 2013

(30) Foreign Application Priority Data
Jul. 28, 2010 (FR) 1056209

(51) Int. Cl. A61K 31/137; A61K 31/4748

(52) U.S. Cl. CPC A61K 31/137; A61K 31/4748
USPC 514/304; 564/361

(57) ABSTRACT

Pharmaceutical composition for the prevention of perioperative arterial hypotension in humans comprising noradrenaline for intravenous administration.

The present invention relates to a novel pharmaceutical composition comprising noradrenaline for the prevention of perioperative hypotension, and specific administration schedules for this composition.

Variations in perioperative and postoperative blood pressure are considered dangerous in elderly patients and “at-risk” patients. Therefore, in the elderly and those being treated for hypertension or presenting aortal stenosis or hemodialyzed patients with renal failure, maintaining blood pressure during anesthesia is a therapeutic priority. Any drop in systolic pressure of 50%, even a brief drop, or of 33% for more than 10 minutes, noticeably increases the mortality rate from cardiac causes during the perioperative period (Goldman L & Caldera D, Risks of general anaesthesia and elective operation in the hypertensive patients, Anesthesiology, 1979, 50(4):285-292).

A French study shows that among the causes of perioperative death imputable to general anesthetic (5.4 per 100,000 anesthesias in France), 39% were the consequence of inadequate perioperative hypotension management (Lienhart A, Auroy Y, Péquignot F et al., Survey of anesthesia-related mortality in France, Anesthesiology 2006; 105: 1087-97).

More recently, a study on 17,067 noncardiac surgical patients showed that the risk of mortality at one year was statistically linked to perioperative hypotension, proportional to the duration of the hypotension (Saager L. et al, Vasopressors May Reduce Mortality Associated with a “Triple Low” of Low Blood Pressure, BIS, and MAC, ASA, Oct. 18, 2009).

Finally, another study has shown the existence of a trend of increasing mortality rate after 1 year with systolic blood pressure (SBP) below 80 mmHg, mean blood pressure (MBP) below 60 mmHg or SBP and diastolic blood pressure (DBP) drops of more than 40-45% of their base values (Bijker J B, van Klei W A, Vergouwe Y et al., Intraoperative hypotension and 1-year mortality after noncardiac surgery, Anesthesiology 2009; 111: 1217-26).

As well as increasing the mortality risk after the operation, the occurrence of perioperative hypotension also causes postoperative side effects to appear. Specifically, it significantly increases the risks of postoperative renal failure in patients (Kheterpal S, Tremper K K, Englesbe MJ, O'Reilly M, Shanks A M, Fetterman D M, et al., Predictors of postoperative acute renal failure after noncardiac surgery in patients with previously normal renal function, Anesthesiology, 2007 December; 107(6):892-902). The occurrence of perioperative hypotension also causes postoperative complications, including noncardiac surgery, such as systemic inflammatory response syndrome (SIRS) or an increased risk of myocardial infarction and cerebral vascular accident in the postoperative period.

In patients, prevention of perioperative hypotension allowing maintenance of blood pressure similar to the initial blood pressure, i.e. before anesthesia, is therefore essential to reducing the health risk and improving the surgical outcomes.

Several methods are used currently to attempt to treat hypotension during anesthesia. Ephedrine and volume expansion are commonly used for perioperative treatment of hypotension.

Ephedrine, always prepared and available on the tray of anesthetic products, is generally the first vasoconstrictor used for hypotension. Ephedrine injected as intravenous 3 to 6 mg bolus is effective quickly. However, it cannot be sustained. Ephedrine allows noradrenaline to be released from the peripheral sympathetic endings, so tachyphylaxis can occur quickly once the nerve endings are depleted of noradrenaline.

Volume expansion, also called vascular filling, is part of the therapeutic arsenal used daily by anesthesiologists. The recommended clinical practice is perfusion of 50 to 200 mL of a colloid or 200 to 500 mL of a crystalloid in 10 to 15 minutes to compensate for vasoplegia caused by the anesthetic. However, the hemodynamic effect is neither immediate, nor easily reversible: the sodium retention that results is responsible for a blood pressure rise during the emergence phase and for delaying the return of normal bowel function after abdominal surgery.

At the date of the present invention, there is no satisfactory treatment that prevents the phenomena of perioperative hypotension, thereby maintaining the blood pressure of the patient at the level where it was before the anesthesia or at a very similar level (less than 40% drop).

Therefore there is a clear need for such a treatment. However, the development of such a treatment was very difficult, as it must:
act immediately on the blood pressure of the patient to act from the start of anesthesia,
deliver a long-lasting effect to last the whole duration of anesthesia,
be perfectly controllable in case of overdose to prevent any major hypertension that could cause irreversible damage,
and finally, have a rapidly reversible effect as soon as it is stopped.

Noradrenaline, or norepinephrine, is an organic neuromediator compound that plays the role of an adrenergic hormone and neurotransmitter. It is a catecholamine like dopamine and adrenaline. It is mainly released by the nerve fibers in the orthosympathetic (or sympathetic) nervous system and acts as a neurotransmitter in the effector organs. It plays a role in attention, emotions, sleep, dreams and learning. It is also the metabolic precursor of adrenaline.

Noradrenaline is sold by various pharmaceutical laboratories in the form of a solution to be diluted for perfusion containing 2 mg/mL of noradrenaline tartrate, i.e. 1 mg/mL of noradrenaline free base.

In intravenous perfusion, noradrenaline is used in resuscitation for emergency treatment of cardiovascular collapse and to restore and maintain blood pressure.

In localized gastric irrigation, noradrenaline is useful for treatment of digestive hemorrhages, as a complement to the usual treatment.

The Summary of Product Characteristics (SPC) recommends initially administering doses of 0.1 to 0.3 μg/kg/min of noradrenaline to the patient. Dosages reaching 3 to 5 μg/kg/min are also recommended in the treatment of septic shock or hemorrhagic shock. Furthermore, combining noradrenaline with a halogen-containing anesthetic gas is not recommended, to prevent serious ventricular rhythm problems.

Using noradrenaline to prevent perioperative hypotension has never been described.

In Intravenous levosimendan-norepinephrine combination during off-pump coronary artery bypass grafting in a hemodialysis patient with severe myocardial dysfunction, Journal of Cardiothoracic Surgery, 2010, 5:9, G. Papadopoulos and colleagues report the combined administration of levosimendan, a positive inotropic agent known to cause hypotension, and noradrenaline to a single patient with heart failure. In the case reported, the doctor decided to administer noradrenaline after the anesthesia induction phase and after the doctor had observed through a transesophageal echocardiogram (TEE) a deterioration in contractility of the myocardium with ventricle dilatation, which required treatment with levosimendan, known to have a side effect of hypotension. Accordingly, for this patient, noradrenaline was administered only to compensate for the hypotensive effects of levosimendan, and not at all to prevent perioperative hypotension.

Furthermore, the French Society for Anesthesia and Resuscitation (Societe Française d'Anesthésie Réanimation, SFAR) recommends against using noradrenaline during anesthesia, stating that adrenaline is the first choice vasoactive agent for cases of severe shock that occur during general anesthesia, and that noradrenaline causes intense vasoconstriction involving the whole of the circulatory system, this effect being dose-dependent. Accordingly, an excessive dose of noradrenaline causes raised vascular resistance and in parallel altered ventricle function with reduced cardiac flow (SFAR, Huet O. & Duranteau J. “Place of vasoactive agents in noncardiac surgery”, Conference on Recent Progress (Conférence d′ actualisation), 1999, p. 161-173).

However, it has been discovered, completely unexpectedly, that the pharmaceutical composition containing noradrenaline according to the present prevents perioperative hypotension with quasi-immediate, long-lasting, controllable and rapidly reversible action.

It has also been discovered that this pharmaceutical composition can be administered concomitantly to prevent perioperative hypotension:
when administering an anesthetic product such as propofol by intravenous route,
and also when administering a halogen-containing anesthetic gas such as sevoflurane without risk of cardiac rhythm problems,
   and regardless of the morphine derivative used (sufentanil, remifentanil, etc.).

Accordingly, the present invention relates to a pharmaceutical composition for the prevention of perioperative hypotension in humans comprising noradrenaline for intravenous administration.

The composition according to the invention maintains the perioperative blood pressure in the patient treated at a level equivalent to that measured before anesthesia.

The effect of the composition according to the invention is quasi-immediate, long-lasting without tachyphylaxis, rapidly reversible as soon as the injection is stopped, and controllable in case of overdose by injection of a nicardipine-type calcium channel blocker such as LOXEN® in bolus at the dose of 1 mg.

The composition according to the invention can be administered for any type of anesthesia, whether using an anesthetic administered by intravenous route such as propofol and/or a halogen-containing anesthetic gas such as sevoflurane, and regardless of the morphine derivative used (sufentanil, remifentanil, etc.).

The composition according to the invention thereby complements, strengthens and improves the “therapeutic arsenal” habitually used and recommended for the prevention of perioperative hypotension, which is the use of ephedrine and volume expansion.

Finally, the composition according to the invention limits, by way of consequences, postoperative side effects associated with the occurrence of perioperative hypotension such as kidney failure, systemic inflammatory response syndrome (SIRS) or the increased risk of myocardial infarction or cerebral vascular accidents in the postoperative period.

In the context of the present invention:
“perioperative hypotension” is understood to mean any systolic blood pressure or mean blood pressure drop phenomenon that could occur during an anesthesia phase, particularly general anesthesia, including in a resuscitation phase, local or regional anesthetic or an epidural, in comparison with the blood pressure of the patient before the anesthesia;
“prevention of perioperative hypotension” is understood to mean maintaining the systolic or mean blood pressure of the patient:
   at a similar value within a limit of 40% of the preoperative systolic or mean blood pressure, and/or
   at a systolic blood pressure greater than or equal to 80 mmHg or at a mean blood pressure greater than or equal to 60 mmHg (Samain E, Pili-Floury S, Barrucand B., Control of the blood pressure of the heart rate in anesthesia 2009 (Contrôle de la pression artérielle de la fréquence cardiaque en anesthésie 2009); 51^{st} National Conference on Anesthesia and Resuscitation. “The Essentials (Les essentiels)”, Edition Elsevier Masson, pages 6 and 7);
the “pharmaceutically acceptable salt” of an active ingredient is understood to mean any addition salt with an inorganic or organic acid of said active ingredient in an organic or aqueous solvent such as an alcohol, a ketone, an ether or a chlorinated solvent, and that is acceptable from a pharmaceutical point of view;
the “pharmaceutically acceptable derivative” of an active ingredient is understood to mean any “prodrug” or “metabolite” of said active ingredient, and their pharmaceutically acceptable salts;
the “prodrug” of an active ingredient is any compound whose biotransformation in the body leads to said active ingredient;
the “metabolite” of an active ingredient is understood to mean any intermediate product resulting from the transformation of said active ingredient in the body during a metabolic process;
noradrenaline (or norepinephrine) designates 4-[(1R)-2-amino-1-hydroxyethyl]benzene-1,2-diol, having chemical structure:

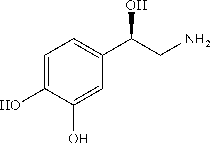 [SMILES: NC[C@H](O)c1ccc(O)c(O)c1]

and its pharmaceutically acceptable salts or derivatives, among which are noradrenaline tartrate, noradrenaline bitartrate and noradrenaline hydrochloride;
ephedrine designates (1RS,2SR)-2-(methylamino)-1-phenylpropan-1-ol, having chemical structure

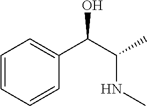

and its pharmaceutically acceptable salts or derivatives, including ephedrine hydrochloride;
atropine designates 8-methyl-8-azabicyclo[3.2.1]oct-3-yl 3-hydroxy-2-phenylpropanoate having chemical structure:

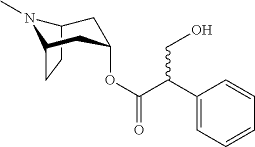

and its pharmaceutically acceptable salts or derivatives, including atropine sulfate;
“continuous administration” is understood to mean administration of a pharmaceutical composition according to a predetermined therapeutic administration schedule that is unlimited and unsequenced or spaced over time, i.e. without treatment interruption;
“bolus administration” is understood to mean intravenous administration, optionally repeated, of a single dose of a pharmaceutical composition.

The present invention therefore relates to a pharmaceutical composition as defined previously to prevent perioperative hypotension in humans. Preferably, the present invention relates to a pharmaceutical composition as defined previously comprising from 0.1 μg/mL to 100 μg/mL of noradrenaline, previously from 0.5 μg/mL to 50 μg/mL of noradrenaline, more preferably from 1 μg/mL to 10 μg/mL of noradrenaline.

The pharmaceutical composition according to the present invention can be formulated in any pharmaceutical form necessary for administration by intravenous route, for instance, a solution to be diluted for perfusion or a ready-to-use solution for perfusion.

The pharmaceutical compositions according to the present invention will therefore comprise, as well as the active ingredient, any pharmaceutically acceptable formulation adjuvant, known to the person skilled in the art and that is necessary to prepare the pharmaceutical composition in the desired form. As an example, sodium metabisulfite, hydrochloric acid, sodium hydroxide or inert gases such as nitrogen or argon can in particular be cited.

The pharmaceutical composition according to the present invention can be administered to any patient under anesthetic or preparing to go under anesthetic. Preferably, the pharmaceutical composition according to the present invention is administered to patients in whom perioperative hypotension will be most pronounced and most harmful, such as the elderly and patients with hypertension or heart disease.

The pharmaceutical composition according the invention therefore prevents perioperative hypotension in humans. The pharmaceutical composition according to the present invention can be administered to the patient according to various administration schedules. However, a particularly effective administration schedule to prevent perioperative hypotension in humans has been discovered for the composition according to the present invention. Accordingly, the present invention relates to a pharmaceutical composition as defined previously, for continuous administration to humans of from 0.005 μg/kg/min to 0.5 μg/kg/min of noradrenaline.

This administration schedule delivers effective, precise and progressive administration of noradrenaline, preventing hypotension without risk of causing harmful hypertension.

Preferably, the present invention relates to a pharmaceutical composition as described previously for continuous administration to humans of 0.01 μg/kg/min to 0.1 μg/kg/min, more preferably from 0.02 μg/kg/min to 0.08 μg/kg/min of noradrenaline.

The pharmaceutical composition according to the present invention can be administered to the patient at any time during anesthesia. Preferably, the composition according to the invention is administered to the patient a few minutes before inducing anesthesia or at the same time and readjusted during the intervention until the patient emerges or the blood pressure rises in parallel with the emergence phase, allowing weaning from noradrenaline and maintaining an optimal pressure. In a particularly preferred manner, the composition according to the invention is administered to the patient from 1 to 5 minutes before inducing anesthesia, even more preferably from 2 to 4 minutes before inducing anesthesia.

The composition according to the present invention can be administered to the patient by any effective means known to the person skilled in the art. In particular, an isolated perfusion can be used to prevent accidental administration of a bolus. To do this, a proximal route can be used of a central venous catheter with several paths, or a different peripheral vein separate from the peripheral venous route used for the anesthetic, or even a three-way tap located directly on the peripheral venous catheter at the end of the perfusion tubing.

The present invention also relates to the use of a pharmaceutical composition as defined previously to prepare a drug intended to prevent perioperative hypotension in humans.

The present invention also relates to the use of a pharmaceutical composition as defined previously for the preparation of a drug intended to prevent perioperative hypotension in humans, said drug being administered continuously by intravenous route so that 0.005 μg/kg/min to 0.5 μg/kg/min, preferably 0.01 μg/kg/min to 0.1 μg/kg/min, more preferably 0.02 μg/kg/min to 0.08 μg/kg/min of noradrenaline are administered to said human.

The present invention also relates to a preventative treatment method for perioperative hypotension in humans by the administration of a pharmaceutical composition as defined previously to said human.

The present invention also relates to a preventative treatment method for perioperative hypotension in humans by continuous administration of a pharmaceutical composition as defined previously to said human, said composition being administered so that 0.005 μg/kg/min to 0.5 μg/kg/min, preferably 0.01 μg/kg/min to 0.1 μg/kg/min, more preferably 0.02 μg/kg/min to 0.08 μg/kg/min, of noradrenaline are administered to said human.

Finally, although the composition according to the present invention could be administered alone, joint, separate or sequenced administration with ephedrine can also be envisaged. Accordingly, the present invention also relates to a pharmaceutical product containing:
a pharmaceutical composition as defined previously; and
a pharmaceutical composition comprising from 0.001 to 0.01 mg/mL, preferably from 0.002 to 0.005 mg/mL of atropine, and/or a pharmaceutical composition comprising from 1 to 10 mg/mL, preferably from 1 to 5 mg/mL of ephedrine;
   as a combination product (or pharmaceutical kit) for simultaneous or separate administration, or administration spread over time for the prevention of perioperative hypotension in humans.

Preferably, the pharmaceutical product according to the present invention contains a pharmaceutical composition as defined previously and a pharmaceutical composition comprising from 0.001 to 0.01 mg/mL, preferably from 0.002 to 0.005 mg/mL of atropine.

In a most preferred manner, the pharmaceutical product according to the present invention allows continuous administration of the pharmaceutical composition according to the invention from 1 to 5 minutes (preferably from 2 to 4 minutes) before anesthesia is induced until the patient emerges, and the separate administration of the pharmaceutical composition comprising atropine in bolus just after the anesthesia is induced.

The present invention is illustrated in a non-limiting manner by the following examples.

### EXAMPLE 1

### Use of Noradrenaline During General Anesthetic and/or Local or Regional Anesthetic in Noncardiac Surgery in Adults

1. Study

The study covered 1,534 patients having received perioperative hemodynamic optimization with use of noradrenaline (in the form of noradrenaline tartrate) to maintain blood pressure under general anesthetic in noncardiac surgery in adults.

2. Experimental Protocol

2-1. Hemodynamic Surveillance

The hemodynamic surveillance is performed non-invasively as blood pressure is taken automatically every 2 minutes when inducing anesthesia, then every 5 minutes during the hemodynamic stability period.

2-2. Therapy

Noradrenaline is used according to the following protocol.

Administration of noradrenaline in continuous perfusion with the solution titered at 10 μg/mL.

This solution is injected:
firstly as a continuous perfusion, in an automatic syringe pump, whose speed will be modulated as a function of the weight, age, and initial “blood pressure state” of the patient (notion of treated or untreated hypertension), of the presence or absence of epidural anesthesia combined with general anesthesia.
secondly as an isolated perfusion to prevent causing a bolus of NA during the injection of another drug in the perfusion tubing. This isolated perfusion is branched either on an extra venous catheter independent of the venous access used for the anesthetic, or on a multi-lumen catheter of a central venous path, or on a three-way tap located between the peripheral venous catheter and the end of the perfusion tubing.

The recommended dosages are 0.02 to 0.08 μg/kg/min of noradrenaline.

The suitable dosage is that which delivers blood pressure similar, within a limit of 20% of the preoperative blood pressure of the patient, and systolic blood pressure >100 mmHg

For general anesthetic, noradrenaline administration by continuous perfusion with the solution titered at 10 μg/mL must be started 3 to 4 minutes before inducing anesthesia to prevent hypotension caused by the anesthetic.

The inefficacy of noradrenaline at these dosages must lead to a search for other etiology for the hypotension: a delay in compensation for perioperative water and electrolyte losses, a caval compression, septic shock, anaphylactic shock.

The patient is weaned off noradrenaline during the emergence phase by progressively reducing noradrenaline administration dosages as a function of how well the patient is returning to the preoperative blood pressure.

3. Study Parameters

3-1. Patients

The study covered 1,534 patients 15 to 102 years old (average age 56 years old), with average weight 72 kg (extremes from 33 to 163 kg), mean preoperative blood pressure 138 mmHg (from 95 to 200 mmHg).

ASA (American Society of Anesthesiologists) Classification: 38.4% of the patients are classed as ASA I (i.e. “normal patient”), 42.2% are classed as ASA II (i.e. “patient with moderate systemic anomalies”), 16.0% are ASA III (i.e. patient with severe systemic anomalies”), and 3.4% are classed as ASA IV.

Age: ranging from 15 to 102 years old; the average age is 56 years old. 25.0% are over 70, including 8.5% who are over 80.

Patient history:
30.1% of patients are smokers (from 15 to 50 packets a year)
32.9% are treated for hypertension
12.6% are treated for heart disease
8.4% are treated for diabetes
0.98% are hemodialyzed patients with renal failure.

### 3-2. Surgeries

The nature of the interventions is varied:
10.6% are orthopedic hemorrhagic surgeries;
18.4% are orthopedic non-hemorrhagic surgeries;
17.9% are laparotomies for digestive, urological or spinal surgery by the anterior route;
11.2% are ENT surgeries;
5.9% are celioscopic surgeries;
12.2% are vascular or thoracic surgeries;
9.3% are plastic or stomatology surgeries;
14.5% are various interventions.

3-3. Anesthetics

The type of anesthesia is varied:
73.8% are isolated general anesthetics;
15.7% are general anesthetics combined with an epidural anesthetic;
9.1% are general anesthetics combined with a local or regional peripheral block;
1.4% are isolated spinal anesthetics without general anesthetic.

The induction procedure for general anesthetic is the same for all the patients: the patients receive 0.2 μg/kg of sufentanil, after 3 minutes 1.5 to 3 mg/kg propofol, then 0.15 mg/kg of cisatracurium.

The patients are intubated 3 minutes after the curare injection and ventilated with 8 mL/kg of theoretical weight.

Anesthesia is maintained using sevoflurane. The perioperative analgesia is handled by continuous injection of remifentanil and/or by iterative injection of sufentanil.

3-4. Noradrenaline Administration

A continuous perfusion of noradrenaline in solution at 10 μg/mL of noradrenaline is administered 4 minutes before inducing anesthesia.

The dosages used are generally comprised between 0.02 and 0.08 μg/kg/min.

Accordingly:
47.9% of the injections are at a dosage comprised between 0.04 and 0.06 μg/kg/min;
22.7% of the injections are at a dosage below 0.04 μg/kg/min in obese patients; and
21.6% of the injections are at a dosage comprised between 0.06 and 0.08 μg/kg/min

Only 7.8% of injections are at a dosage comprised between 0.08 and 1 μg/kg/min in very old patients, or hemodialyzed patients with renal failure.

The mean duration of administration of the continuous perfusion of noradrenaline was 105 min (extremes from 15 to 420 min), from inducing anesthesia to weaning in the emergence room.

The patient is weaned off the continuous perfusion of the noradrenaline solution at 10 μg/mL in the emergence phase by progressively reducing the noradrenaline administration dosages as a function of how well the patient is returning to the preoperative blood pressure.

In all cases, weaning occurred without incident and quickly in the emergence phase.

In 1.4% of cases the weaning was only done after vascular filling, meaning a delay in vascular filling.

3-5. Detection of Undesirable Effects

Clinically: non-invasive blood pressure surveillance is performed every 2 minutes when inducing anesthesia, then every 5 minutes during the hemodynamic stability period.

The quality of the perioperative hemostasis was always noted. As long as the blood pressure does not exceed the initial blood pressure of the patient, no excessive perioperative bleeding is noted.

The following elements are systematically monitored and noted in the first 24 hours postoperative: agitation when waking, postoperative nausea and/or vomiting (PONY).

Biologically: troponin was assayed at D1 in 26.0% of patients judged to be at risk: 99.87% were at zero, one patient has a troponine assay at 0.04 and one patient at 0.05 μg/mL (significance threshold is 0.4 μg/mL). These patients were controlled again and their level had normalized at D2.

Urea and creatinine assay on D0 and D1 performed on 26.0% of the patients judged to be at higher risk. There were no changes to renal function from the results.

Radiologically: a venous Doppler of the lower members is performed in at-risk patients at D4 (26.0% of patients): five cases of superficial venous thromboses were detected.

4. Experimental Results

4-1. Classed by Patient Type

In elderly patients the risk is high: maintaining blood pressure is a therapeutic priority. Noradrenaline was first used as an anesthetic in these patients with the goal of safety: a drop in systolic blood pressure of 50%, even brief, or of 33% for more than 10 minutes, increases mortality due to heart-related problems during the perioperative period. A study on 17,067 noncardiac surgical patients showed that the risk of mortality at one year was statistically linked to perioperative hypotension, proportional to the duration of the hypotension (Saager L. et al, Vasopressors May Reduce Mortality Associated with a “Triple Low” of Low Blood Pressure, BIS, and MAC, ASA, 2009, 50(4):285-292).

8.5% of the patients in the study are over 80 years old, the health risk was controlled and surgical outcomes were simple from the neurological, cardiac and respiratory standpoint. According to this study, the noradrenaline dosages used in very elderly patients to maintain blood pressure similar to the initial blood pressure are higher than the dosages used in younger patients.

In treated hypertensive patients: the notion of prevention and correction of hypotension is even more important. According to this study, the noradrenaline dosages used to maintain blood pressure similar to the initial blood pressure are higher than the dosages used in patients without hypertension.

In patients with aortal stenosis: five cases of elderly patients with aortal stenosis and hip fracture, using noradrenaline controlled perioperative blood pressure during general anesthesia without cardiac incidents.

In hemodialyzed patients with renal failure: they have cumulative cardiovascular risk factors and for these patients, volume filling should be avoided, noradrenaline injection meant blood pressure could be controlled, but with higher administration dosages: 0.05 to 0.08 μg/kg/min

In young patients without history: perioperative blood pressure changes are considered as well tolerated, but low doses of noradrenaline 0.02 μg/kg/h are enough to optimize blood pressure and lead to higher quality emergence: better lucidity and quasi-disappearance of postoperative nausea and vomiting.

4-2. Classed by Intervention Type

Interventions by celioscopy: noradrenaline injection, started when inducing anesthesia, was reduced then stopped as blood pressure increased as the celioscopic insufflation was inserted, apart from in a few cases (12.5%) where the noradrenaline doses were reduced but maintained at low dosages (0.025 to 0.04 μg/kg/min) during the celioscopy.

Hemorrhagic surgery: in this study the noradrenaline injection at these dosages and the maintenance of blood pressure similar to initial blood pressure did not increase perioperative bleeding as long as the level of anesthesia and analgesia are sufficiently high. In itself, being able to maintain a high enough level of anesthesia and analgesia without risk of hypotension seems to be a guarantee of less perioperative bleeding. In case of massive blood loss, the use of noradrenaline perfusions means a perfusion pressure can be maintained while waiting for a volume expansion suitable for the blood loss.

Case of laparoscopy: the combination of general anesthetic and epidural anesthetic increases the risk of perioperative hypotension. At these dosages, the noradrenaline injection controlled the blood pressure in a prolonged manner (up to 400 min) without tachyphylaxis and without excess vascular filling: many studies advocate reducing perioperative volume additions, which seems to reduce the delay in normal bowel function resuming, shortens hospitalization and reduces cardiac and infectious complications.

The possibility of preventing perioperative hypotension improves the level of anesthesia without risk of creating hypotension, thereby preventing under-dosages in anesthesia responsible for perioperative hypertension.

### 4-3. Undesirable Effects

Hypertension: hypertension must not be able to occur at the recommended dosages. It is more connected to insufficient anesthesia and analgesia. When faced with the possibility of hypertension, the measures taken were respectively:
stronger anesthesia and analgesia,
reducing the rate of noradrenaline administration (brief duration of action),
and, if necessary, as a last resort, use vasodilation drugs of the calcium-channel inhibitor type: NICARDIPINE (LOXEN®) at the dose of 1 mg as a bolus (26 cases).

Bradycardia: using a vasoconstrictor activates the baroreceptor reflex system, meaning bradycardia. This bradycardia is increased in patients treated with betablockers and in patients whose analgesia was a REMIFENTANIL (ULTIVA®) type morphine.

Atropine is used as a preventive measure in practice (in 90% of cases in our study) as soon as the pulse of the patient begins to slow, at the end of inducing anesthesia.

Renal function: this is a theoretical risk for noradrenaline at the dosages usually used in resuscitation. In contrast, in the scope of the stress at the dosages proposed for anesthesia, in all patients at potential renal risk, urea and creatinine measurements at D0 and D1 showed in all cases a reduction of their postoperative value at D1. A net increase in perioperative urine flows was noted, which means good renal function.

No other undesirable effect has been shown during the study.

Specifically, the absence of morbidity is a safety guarantee.

4-6. Advantages

Blood pressure maintenance: blood pressure maintenance is the goal of this study for noradrenaline use; it is a safety guarantee in the elderly, and in patients with hypertension or heart disease.

The effect of noradrenaline on blood pressure is immediate, brief, but without tachyphylaxis, long-lasting in continuous injection, rapidly reversible.

Alternative to vascular filling: vascular filling must be able to remain moderated to prevent any detrimental overload. The absence of morbidity after using noradrenaline in the scope of this study does make it a good alternative to vascular filling.

Postoperative nausea and vomiting (PONY): none of the patients with blood pressure controlled by noradrenaline injection suffered from vomiting. The little nausea (1.2% versus 30% traditionally) was controlled by ZOPHREN. Some patients with a history of PONY (18%) were pleased to observe that they had not had PONY this time.

Lucidity at emergence and absence of agitation: were noted in all patients, independent of duration of intervention and patient age.

Venous Thromboembolism Disorders: only 0.32% of distal peripheral venous thrombosis complications versus 5 to 40% traditionally depending on the type of surgery. The absence of thromboembolic complications is notable. A venous Doppler is systematically taken at D4 in at-risk patients. The thromboembolic risk is reduced without the hemorrhagic risk being increased.

4-7. Conclusion

The noradrenaline composition according to the invention thereby complements, strengthens and improves the “therapeutic arsenal” habitually used and recommended for the prevention of perioperative hypotension, which is the use of ephedrine and volume expansion.

### EXAMPLE 2

### Practical Use of the Noradrenaline Composition According to the Present Invention

The following table is a practical example of the use of the noradrenaline composition (in the form of noradrenaline tartrate) according to the present invention that allows the administration rate from the syringe (in mL/h) containing the noradrenaline solution at 10 μg/mL to be determined as a function of the quantity of noradrenaline administered (in μg/kg) and the weight of the patient (in kg).

| Quantity | | | | | | | | | | | |
|---|---|---|---|---|---|---|---|---|---|---|---|
| of noradrenaline | Patient weight (in kg) | | | | | | | | | | |
| tartrate (in μg/kg) | 50 | 55 | 60 | 65 | 70 | 75 | 80 | 85 | 90 | 95 | 100 |
| 0.02 | 6 | 6.6 | 7.2 | 7.8 | 8.4 | 9 | 9.6 | 10.2 | 10.8 | 11.4 | 12 |
| 0.03 | 9 | 9.9 | 10.8 | 11.7 | 12.6 | 13.5 | 14.4 | 15.3 | 16.2 | 17.1 | 18 |
| 0.04 | 12 | 13.2 | 14.4 | 15.6 | 16.8 | 18 | 19.2 | 20.4 | 21.6 | 22.8 | 24 |
| 0.05 | 15 | 16.5 | 18 | 19.5 | 21 | 22.5 | 24 | 25.5 | 27 | 28.5 | 30 |
| 0.06 | 18 | 19.8 | 21.6 | 23.4 | 25.2 | 27 | 28.8 | 30.6 | 32.4 | 34.2 | 36 |
| 0.07 | 21 | 23.1 | 25.2 | 27.3 | 29.4 | 31.5 | 33.6 | 35.7 | 37.8 | 39.9 | 42 |
| 0.08 | 24 | 26.4 | 28.8 | 31.2 | 33.6 | 36 | 384 | 40.8 | 43.6 | 45.6 | 48 |

1. A pharmaceutical composition for the prevention of perioperative hypotension in humans comprising noradrenaline for intravenous administration.

2. The pharmaceutical composition according to claim 1, characterized in that it comprises from 0.1 μg/mL to 100 μg/mL of noradrenaline.

3. The pharmaceutical composition according to claim 2, characterized in that it comprises from 0.5 μg/mL to 50 μg/mL of noradrenaline.

4. The pharmaceutical composition according to claim 3, characterized in that it comprises from 1 μg/mL to 10 μg/mL of noradrenaline.

5. The pharmaceutical composition according to claim 1, for continuous administration to humans of from 0.005 μg/kg/min to 0.5 μg/kg/min of noradrenaline.

6. The pharmaceutical composition according to claim 5, for continuous administration to humans of from 0.01 μg/kg/min to 0.1 μg/kg/min of noradrenaline.

7. The pharmaceutical composition according to claim 6, for continuous administration to humans of from 0.02 μg/kg/min to 0.08 μg/kg/min of noradrenaline.

8. The pharmaceutical composition according to claim 1, characterized in that the composition is administered to the patient from 1 to 5 minutes before anesthesia is induced until the patient emerges.

9. A pharmaceutical product containing:
a pharmaceutical composition as defined in claim 1; and
a pharmaceutical composition comprising from 0.001 to 0.01 mg/mL of atropine and/or a pharmaceutical composition comprising from 1 to 10 mg/mL of ephedrine;
as a combination product for simultaneous or separate administration, or administration spread over time for the prevention of perioperative hypotension in humans.

10. The pharmaceutical product according to claim 9, containing a pharmaceutical composition as defined in claim 1 and a pharmaceutical composition comprising from 0.001 to 0.01 mg/mL of atropine.

11. The pharmaceutical product according to claim 10, for continuous administration of the composition as defined in claim 1 from 1 to 5 minutes before anesthesia is induced until the patient emerges, and the separate administration of the pharmaceutical composition comprising atropine in bolus just after the anesthesia is induced.
